(19) United States
(12) Patent Application Publication
(10) Pub. No.: US 2005/0085452 A1
(43) Pub. Date: Apr. 21, 2005

(54) Process for the synthesis of pharmacologically active (z/e)-guggulsterones

(76) Inventors: Ganga Raju Gokaraju, Andhra Pradesh (IN); Rama Raju Gokaraju, Andhra Pradesh (IN); Venkata Subbaraju Gottumukkala, Andhra Pradesh (IN); Venkateswarlu Somepalli, Andhra Pradesh (IN)

Correspondence Address: RADER FISHMAN & GRAUER PLLC, LION BUILDING, 1233 20TH STREET N.W., SUITE 501, WASHINGTON, DC, 20036, US

(21) Appl. No.: 10/498,316

(22) PCT Filed:
(86) PCT No.: PCT/IN02/00181

(51) Int. Cl. A61K 31/57; C07J 5/00; C07J 7/00

(52) U.S. Cl. 514/177; 552/582

(57) ABSTRACT

The invention relates to an improved process for producing pharmacologically active synthetic stereoisomeric mixture of guggulsterones (4) in the three steps. The mixture of gugguisterones consists of Z-guggulsterone [4,17(20)-trans-pregnadiene-3,16-dione] and E-guggulsterone [4,17(20)-cis-pregnadiene-3,16-dione] and could be in any relative ratio. This improved process comprises (a) epoxidation of 16-dehydropregnanolone acetate (1) with hydrogen peroxide (b) reduction of the so obtained epoxide (2) with hydrazine hydrate and (c) oxidation of the diol (3).

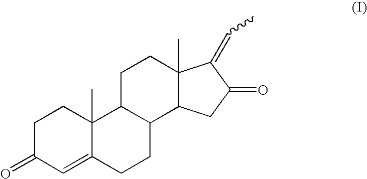

### TECHNICAL FIELD OF THE INVENTION

This invention provides a novel process for the synthesis of pharmacologically active Z/E stereoisomeric mixture of guggulsterones in high purity. Guggulsterones consists of a mixture of Z-guggulsterone, which is 4,17(20)-trans-pregnadiene-3,16-dione and E-guggulsterone, which is 4,17(20)cis-pregnadiene-3,16-dione. This mixture of stereoisomeric pregnadiene-3,16-diones may be in any relative ratio and is herein after referred as (Z/E)-guggulsterones.

### BACKGROUND OF THE INVENTION

Guggul resin obtained from the tree Commiphora mukul belonging to the family Burseraceae is known as guggulu in Sanskrit and guggul in Hindi. Indian Patent Specification No. 148265 describes a process for extraction of guggulipid from guggul resin by ethyl acetate. Guggulipid is found to contain two stereoisomers of guggulsterones. Medicinal activity of the plant extract is similar to the well known drug clofibrate but without its side effects.

Isolation of Z and E-guggulsterones from the oleo-resin or gum of the tree Commiphora mukul has also been reported in Tetrahedron, 1972, 28, 2341-52 by Sukh Dev, et al. A process for obtaining pharmacologically active fraction, guggulipid, has also been disclosed in the U.S. Pat. No. 5,273,747, 1993. Guggulsterones have been shown to exhibit beneficial action in the management of ischaemic heart diseases, hypolipidaemic and anti-inflammatory action with no side effects.

However, the tree Commiphora mukul having the biologically active lipid is not easily available and a large number of trees are to be harvested for the resin. The naturally occurring resin has only a low concentration of the active guggulsterones. Further, preparation of dosage forms such as tablets or capsules from the oleo-resin extract poses problems due to its gummy nature.

European patent No. EP. 0 447 706, discloses a four step procedure for the synthesis of Z and E-guggulsterones. This process includes the reduction of an α,β-unsaturated ketone of 16-dehydropregnenolone acetate (16-DPA) with lithium aluminium hydride in dry tetrahydrofuran. The second step is an acid catalysed isomerisation using acetic anhydride, acetic acid and P-toluene sulphonic acid. The reagents and solvents used in this process are expensive and the process is tedious.

The object of this invention is to provide an improved process for the production of pharmacologically active synthetic Z/E-guggulsterones, which is cost effective. The product obtained has high purity.

### DISCLOSURE OF THE INVENTION

The present invention is an improved process for the preparation of pharmacologically active synthetic Z/E-guggulsterones in three steps. The process comprises the reaction of 16-dehydropregnenolone acetate with hydrogen peroxide in a polar solvent in the presence of a base to give 16,17-epoxy-3-hydroxy-5-pregnen-20-one. This is subjected to Wolff-Kishner reduction and elimination under Huang-Minlon conditions with hydrazine hydrate in a polar solvent and/or in the presence of a base to give a diastereomeric mixture of 5,17(20)pregnadiene-3,16-diol. Oppenauer oxidation of the diol mixture in an aromatic solvent in the presence of a catalyst using a hydrogen acceptor produces Z/E-guggulsterones, which is isolated and subjected to further purification.

This invention relates to a process for synthesising pharmacologically active Z/E-guggulsterones which comprises reacting 16-dehydropregnenolone acetate with hydrogen peroxide in the presence of a base to give the corresponding epoxide, reducing said epoxide with hydrazine hydrate to produce a diastereomeric mixture of 5,17(20)pregnadiene-3,16-diol, oxidizing said diastereomeric mixture with a hydrogen acceptor in the presence of a catalyst to produce Z/E-gugguisterones which is isolated and purified in a known manner.

The reaction in the first two stages may be carried out in a polar solvent such as methanol, ethanol, isopropyl alcohol t-butanol and mixtures thereof. Non-polar solvents such as tetrahydrofuran, dioxane, ether and mixtures thereof may also be used. Aromatic solvents such as toluene, xylenes and chlorobenzene may also be used in the oxidation step. Catalyst used in the reaction may be selected from aluminium isopropoxide, aluminium isobutoxide, aluminium phenoxide or aluminium tertiary butoxide.

The hydrogen acceptor in the oxidation stage may be acetone, ethyl methyl ketone, diethyl ketone, methyl isobutyl ketone, cyclopentanone, cyclohexanone, cycloheptanone, acetophenone or benzophenone.

Purification of the diastereomeric mixture of guggulsterones is carried out by chromatographic separation on a silica gel column using polar and non-polar solvents or mixtures thereof. Reversed phase silica column separation may also be used for purifying the product. The polar solvents used for elution are ethanol, methanol or water and non-polar solvents include hexane, cyclohexane, toluene, chloroform, ethyl acetate or acetone and mixtures thereof.

The reaction scheme is shown below.

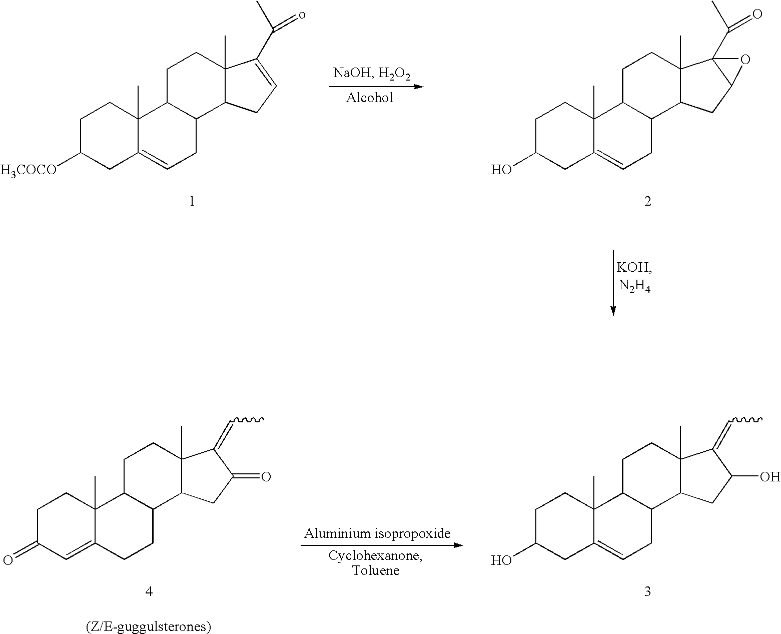

The following example illustrates the present invention but do not limit the scope thereof.

### Preparation of 16,17-epoxy-3-hydroxypregn-5-en-20-one (2)

To a solution of 16-dehydropregnenolone acetate (4.0 Kg) in alcohol (100 L) was added hydrogen peroxide (1.6 L) followed by dropwise addition of 4N sodium hydroxide solution for 1 hr. The reaction mixture was stirred at 5-15° C. for 12 hrs and the solid obtained was filtered and dried to give compound of formula 2 in the reaction scheme (Yield: 3.52-3.66 Kg, 95-99%, m.p. 194-196° C.).

### Preparation of 5,17(20)-pregnadiene-3,16-diol (3) from 16,17-epoxy-3-hydroxypregn-5-en-20-one

To a solution of the ¹6,17-epoxy-3-hydroxypregn-5-en-20-one (2.0 Kg) in hydrazine hydrate (7.2 L) was added potassium hydroxide (0.7 Kg) and the mixture was heated for 4 hrs. The cooled reaction mixture was diluted with ice cold water and acidified with dilute HCl. The solid obtained was filtered and washed with water and dried to give the compound of formula 3 shown in the reaction scheme (yield: 1.53-1.82 Kg, 80-95%, m.p. 168-172° C.).

### Preparation of 5,17(20)-pregnadiene-3,16-diol (3) from 16,17-epoxy-3-hydroxypregn-5-en-20-one

Alternate Route:

To a solution of the 16,17-epoxy-3-hydroxy pregn-5-en-20-one (91 g) in alcohol (2.0 L) was added hydrazine hydrate (155 mL) and the mixture was refluxed for 6 hrs. Solvent was removed at reduced pressure and diluted with cold water. The solid separated was filtered, washed with cold water and dried to give compound of the formula 3 shown in the reaction scheme (yield: 69.7-82.8 g, 80-95%, m.p. 168-170° C.).

### Preparation of 4,17(20)-pregnadiene-3,16-dione (4) from 5,17(20)-pregnadiene-3,16-diol

A solution of 5,17(20)-pregnadiene-3,16-diol (0.8 Kg) in toluene (30.0 L), cyclohexanone (4.0 L) and aluminium isopropoxide (0.75 Kg) were refluxed for 4 hrs. The reaction mixture was cooled, acidified with 10% sulfuric acid (5.0 L) and separated the layers. The aqueous layer was extracted twice with toluene (2.0 L). The combined toluene layer was washed sequentially, with water (2.0 L), 10% NaHCO₃ (2.0 L) and water (2.0 L). Toluene was distilled off under vaccum to give a gummy residue, which was chromatographed over silica gel column eluting using mixtures of pet.ether-ethyl acetate for elution to give the Z/E-guggulsterones (Yield: 0.39-0.71 Kg, 50-90%, m.p. 174-178° C.). The ratio of Z to E in the final product was found to be in the range of 10 to 0.1 and the purity of gugguisterones is >99% by HPLC.

1. A process for synthesizing pharmacologically active Z/E guggulsterones which comprises reacting 16-dehydropregnenolone acetate with hydrogen peroxide in the presence of a base to give the corresponding epoxide, reducing said epoxide with hydrazine hydrate to produce a diastereomeric mixture of 5,17(20)-pregnadiene-3,16-diol, oxidizing said diastereomeric mixture with a hydrogen acceptor in the presence of a catalyst to produce Z/E-guggulsterones which is isolated and purified in a known manner.

2. The process in accordance with claim 1 wherein hydrogen peroxide in the concentration of 30 to 50% is added to 16-dehydropregenolone acetate in a polar solvent.

3. The process in accordance with claim 2 wherein said polar solvent is selected from methanol, ethanol, isopropyl alcohol or t-butyl alcohol and mixtures thereof.

4. The process in accordance with claim 1 wherein the reaction between 16-dehydropregnenolone acetate with hydrogen peroxide is carried out in the presence of a base such as sodium hydroxide, potassium hydroxide or other alkaline metal hydroxides.

5. The process in accordance with claim 1 wherein said epoxide is reduced with hydrazine hydrate in a polar solvent or in the presence of a base such as sodium hydroxide, potassium hydroxide or other alkaline metal hydroxides.

6. The process in accordance with claim 5 wherein said polar solvent is selected from methanol, ethanol, isopropyl alcohol or t-butyl alcohol and mixtures thereof

7. The process in accordance with claim 1 wherein said diastereomeric, mixture of 5,17(20)-pregnadiene-3,16-diol is oxidised in an aromatic solvent with a hydrogen acceptor in the presence of a catalyst.

8. The process in accordance with claim 7 wherein said aromatic solvent is selected from toluene, xylenes and chlorobenzene.

9. The process in accordance with claim 7 wherein the catalyst used in the third stage is selected from aluminium isopropoxide, aluminium isobutoxide, aluminium phenoxide and aluminium tertiary butoxide.

10. The process in accordance with claim 7 wherein said hydrogen acceptor is selected from acetone, ethyl methyl ketone, diethyl ketone, methyl isobutyl ketone, cyclopentanone, cyclohexanone, cycloheptanone, acetophenone or benzophenone.

11. The process in accordance with claim 1 wherein said diastereomeric mixture of guggulsterones is purified by silica gel column chromatography using polar and non polar solvents or mixtures thereof, as eluents.

12. The process in accordance with claim 1 wherein said diastereomeric mixture of guggulsterones is purified by reversed phase silica gel column chromatography using polar solvents as eluents.

13. The process in accordance with claim 11 wherein said polar solvents are selected from methanol, ethanol, isopropyl alcohol or water and said non-polar solvents are selected from petroleum ether, hexane, cyclohexane, toluene, chloroform, ethyl acetate or acetone and mixtures thereof.

14. Pharmacologically active Z/E-guggulsterones whenever prepared by a process according to claim 1.
